(19) United States
(12) Patent Application Publication
(10) Pub. No.: US 2010/0037366 A1
(43) Pub. Date: Feb. 18, 2010

(54) HAT FOR SPECIFIC AREA APPLICATION OF HEAT OR COLD

(76) Inventor: Timothy Panicali, Elma, NY (US)

Correspondence Address: PATRICIA M. COSTANZO;PATS PENDING, P.O. BOX 101, ELMA, NY, 14059, US

(21) Appl. No.: 12/540,725

(22) Filed: Aug. 13, 2009

Related U.S. Application Data
(60) Provisional application No. 61/188,767, filed on Aug. 13, 2008.

(51) Int. Cl. A61F 7/00; A42C 99/00; A42B 1/24

(52) U.S. Cl. 2/171.2; 2/209.13; 607/109

(57) ABSTRACT

A hat having exterior and interior layers connected to each other only along the periphery of the hat creating an unobstructed interlayer space for insertion of a thermally retentive pack(s) providing for the application of heat or cold to any specific area or all of a user's head, ears, and forehead. The layers and space sized and shaped to fit about the contours of a user's entire scalp, ear, and forehead area. The interlayer space is bounded by the inner exterior and inner interior layer surfaces and a seam (forms??) from the layer's connection, and has no seams creating barriers to the exact positioning of a hot or cold pack where it is required. There is a slit throughout the interior and/or exterior layer through which a specific spot-sized or whole head-sized hot or cold pack is inserted. There is an optional strap for securing the hat about a head.

### CROSS-REFERENCE TO RELATED APPLICATIONS

This application claims the benefit of Provisional Application No. 61,188,767 filed Aug. 13, 2008

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND

The present invention relates generally to means for applying heat or cold to a user's head, and more particularly to a hat designed to provide heat or cold to any specific desired area of a user's head, including ears, and forehead or for full head treatment.

The background information discussed below is presented to better illustrate the novelty and usefulness of the present invention. This background information is not admitted prior art.

When a person hits their head on a relatively soft surface, as a result of falling of otherwise, the height of the fall is less than the height of the person who fell, and there is no loss of consciousness; seeing a doctor is usually not required. To help reduce both swelling and pain, the immediate application of ice to the injured area is recommended. The application of ice at first will constrict local blood vessels and decrease tissue temperature in order to decrease both blood flow and cell metabolism, which limits hemorrhaging and cell death. After about twenty minutes of treating the injured area with ice, blood vessels will slowly dilate and tissue temperature will increase. Local therapeutic cold applications also helps to manage edema, muscle spasm, bleeding, and pain. The healing process, however, requires increased blood flow into the injured area to support the process of cellular growth. As long as swelling persists and circulation is congested, the healing process is delayed or retarded. The swelling must be reduced before full recovery can occur, thus, for the first 72 hours after an injury the application of cold and the use of compression are medically recommended therapies to help reduce local pain and swelling. More importantly, by containing the severity of swelling, the application of cold and compression encourages healing. However, once swelling has subsided, the application of heat helps reduce pain and promote healing.

There are other instances where the application of heat, instead of, or in addition to, the application of cold, is recommended. For example, one of the most popular and simple remedies for an earache is to apply heat to the affected ear for three to five minutes at a time to ease the sometimes excruciating pain of an earache. This can be accomplished by either by placing a heating pad wrapped in a towel upon the ear, or by directing a hair dryer set to low heat into the ear canal from a distance of eight to 12 inches for about three minutes at a time. Heat is also applied to a person's head when their hair is being coloring or moisturized. Heat treatments, focused on the head are also required for the penetration of hair and scalp treatments, such as for hair straightening.

### SUMMARY

At the heart of the present invention is applicant's discovery that there was no way to easily or effectively apply either heat or cold to specific areas of a person's head because there is no product that provides for targeting the placement of a heat or cold pack exactly and only where it is required, and then to maintain the pack over the injured area. Presently available products come with a series of isolated pockets in which a thermal pack may be inserted. However, the seams between the pockets often are positioned exactly where the treatment is required and, thus, prevent targeted treatment. Moreover, there is no means available to apply heat or cold over the ear or the forehead area.

In accordance with the principles of applicant's invention, there is provided a hat having an unobstructed, single, accessible interlayer space shaped to cover the entire surface area of a head, including the ear and forehead area. The principles of the invention include having a hat made of an outer layer of material and an inner layer of lining where the lining is connected to the hat's outer layer of material only about the periphery of the hat, thus forming an accessible, unimpeded interlayer space contouring the entire head surface area including the ear and forehead areas. If desired, the invention also contemplates providing a mechanism for affixing the hat to a head, such as a tie or strap. A slit in the lining provides an opening through which a hot or cold pack is inserted into the unimpeded interlayer space. The thermal retentive packs provided with the hat are much smaller than a head's surface area so as to be manually positioned between the lining and the outer material of the hat directly over the injured or painful area, including the forehead and ear area. This provides for the therapeutic effects of the thermal pack to be precisely targeted. For example, if a person has an earache in his right ear, the pack is placed in the interlayer space between the lining and the outer material so that it will be directly over the ear when the hat with the pack is placed on the person's head. This protects the user from the discomfort, or even from the detrimental effects, of having the user's entire head cooled or heated, when only a specific area requires thermal treatment. The inner surface of the outer material and the inner surface of the lining that is facing the inner surface of the outer material are made from a material that is provided with the amount of roughness required for the pack to be securely held in place solely by the frictional bond between the outer surfaces of the pack and the inner surfaces of the hat's outer material and its lining.

As a parent of a young, active child who has several times experienced minor bumps and bruises on various areas of his head, including his forehead, requiring the application of cold and who has suffered from painful ear infections requiring the application of heat, applicant soon realized how difficult it is to keep a cold or hot pack on the area requiring treatment on a child's head. Some children are too young to be given the responsibility of holding the pack in place and older children, in similar circumstances, complain that their arm hurts from holding the pack on their head. Thus, the principles of applicant's invention provide for a hat having an unobstructed, single, interlayer space that covers the entire surface area of a head, including the ear and forehead area, and that will securely maintain a hot or cold pack in exactly the position that is required to treat the area requiring treatment without assistance. Moreover, if a full head treatment were required, such as when a person is having his or her hair colored, or after a head operation, head packs shaped to mimic the shape of a human skull are also available and can be easily and rapidly inserted into the single, unobstructed, interlayer space that covers the entire surface area of a head for easy and effective full-head treatment. Additionally, a hat made by the principles of the present invention is useful when a child, or adult, is outdoors on a cold, windy day; the hat with a hot pack inserted keeps the user warm for a significantly extended amount of time, as it is well known that the head is one of the major conductors of body heat.

The hat may be used by a child or by an adult, as offering hats of various styles and sizes is within the contemplation of the invention. A hat made using the principles of this invention may be stylized to look like an animal, a clown, or be simple and undecorated, as long as the hat does not provide multiple chambers, the seams of which would impede the placement of the thermal pack exactly where required.

A hat according to the principles of the present invention is taught by providing for a hat, comprising:

a hat made of two layers of material, comprising
an exterior layer sized and shaped to fit about the contours of a head,
an interior layer sized and shaped to fit about the contours of a head,

the exterior layer connected to the interior layer only along the periphery of the hat creating an unobstructed interlayer space for the insertion of a thermally retentive pack providing for the application of heat or cold to any specific area of a user's head, ear, and forehead.

Furthermore, wherein the hat is shaped so as to cover the user's entire scalp, ear, and forehead area and the interlayer space is shaped so as to cover the user's entire scalp, ear, and forehead area.

Still furthermore, wherein the exterior layer further comprises
an exterior layer outer surface area and an exterior layer inner surface area, and
the interior layer further comprises
an interior layer outer surface area and an interior layer inner surface area,
   so that the unobstructed interlayer space is bounded by the inner exterior layer surface area, the inner interior layer surface area, and a seam resulting from the exterior layer connected to the interior layer only along the periphery of the hat.

Yet, still, wherein the exterior layer is connected to the interior layer by sewing and/or the exterior layer is connected to the interior layer by adhesive bonding.

Furthermore, wherein the exterior layer is connected to the interior layer so as ensure that the interlayer space created by the connection has no seams creating barriers to the exact positioning of a hot or cold pack where it is required over a user's scalp, ear, or forehead area.

And, wherein the hat further comprises a strap for securely affixing the hat to a user's head.

Moreover, wherein the hat further comprises a slit throughout the interior layer so as to provide an opening through which a hot or cold pack is inserted into the interlayer space, or wherein the hat further comprises a slit throughout the exterior layer so as to provide an opening through which a hot or cold pack is inserted into the interlayer space, or wherein the hat further comprises a slit throughout the exterior layer and throughout the interior layer so as to provide an opening through which a hot or cold pack is inserted into the interlayer space.

Wherein the hat further comprises at least one thermal retentive pack for insertion into the interlayer space, the pack shaped and sized to be applied over a specific area of a head requiring treatment and, or the hat further comprises at least one thermal retentive pack for insertion into the interlayer space, the pack shaped and sized to be applied over a user's whole head area.

It is also contemplated that the hat further comprises being made so as to provide for combined hat and thermal retentive pack cooling.

Another favored embodiment, envisions a hat, comprising:

a hat made of two layers of material, comprising
an exterior layer sized and shaped to fit about the contours of a head,
an interior layer sized and shaped to fit about the contours of a head,

the exterior layer sewn to the interior layer only along the periphery of the hat creating an unobstructed interlayer space for the insertion of a thermal retentive pack providing for the application of heat or cold to all specific areas of a user's head, ear, and forehead areas,

the hat shaped so as to cover the user's entire scalp, ear, and forehead area,

the interlayer space shaped so as to cover the user's entire scalp, ear, and forehead area, having

a slit throughout the interior layer and/or the exterior layer so as to provide an opening through which a thermally retentive pack is inserted into the interlayer space and having,

a strap for securely affixing the hat to a user's head.

Still yet another embodiment, contemplates a hat in combination with at least one thermally retentive pack, the hat made of two layers of material, comprising

an exterior layer sized and shaped to fit about the contours of a head,

an interior layer sized and shaped to fit about the contours of a head,
the exterior layer sewn to the interior layer only along the periphery of the hat creating an unobstructed interlayer space for the insertion of a thermal retentive pack,
the hat shaped so as to cover the user's entire scalp, ear, and forehead area,
the interlayer space shaped so as to cover the user's entire scalp, ear, and forehead area,
the hat having
a strap for securely affixing the hat to a user's head, and
a slit throughout the interior layer and/or the exterior layer so as to provide an opening through which the thermally retentive pack is inserted into the interlayer space providing for the application of heat or cold to all specific areas of a user's head, ear, and forehead areas, wherein the hat further comprises a thermally retentive pack sized and shaped to conform to a user's entire scalp area.

Still other benefits and advantages of this invention will become apparent to those skilled in the art upon reading and understanding the following detailed specification and related drawings.

### BRIEF DESCRIPTION OF THE DRAWINGS

In order that these and other objects, features, and advantages of the present invention may be more fully comprehended and appreciated, the invention will now be described, by way of example, with reference to specific embodiments thereof which are illustrated in appended drawings wherein like reference characters indicate like parts throughout the several figures. It should be understood that these drawings only depict preferred embodiments of the present invention and are not therefore to be considered limiting in scope, thus, the invention will be described and explained with additional specificity and detail through the use of the accompanying drawings, in which:

FIG. 1 is a perspective view of a hat made according to the principles of the present invention.

FIG. 2 is a perspective view of the hat, as shown in FIG. 1, being worn by a child.

FIG. 3 is a perspective view of the inside of the hat, as shown in FIG. 1.

FIG. 4 is a perspective view of examples of thermo-retentive packs that are used in conjunction with a hat of the present invention.

FIG. 5a is an elevation view of a full scalp covering thermo-retentive pack.

FIG. 5b is a perspective view of a full scalp covering thermo-retentive pack.

### A LIST OF THE REFERENCE NUMBERS AND THE PARTS TO WHICH THEY REFER

10 A hat following the principles of the present invention.
12 A strap for securing hat 10 on a head.
12a Connector for connecting strap to hat 10.
14 A exterior material layer of hat 10.
14a A outer surface of exterior layer 14 of hat 10.
14b An inner surface of exterior layer 14 of hat 10.
16 An interior material layer, or lining, of hat 10.
16a A outer surface of interior material layer, or lining, 16 of hat 10.
16b An inner surface of interior material layer, or lining, 16 of hat 10.
18 Ear covering part of hat 10.
20 Forehead covering part of hat 10.
22 Seam about the periphery of the hat connecting the hat's outer layer of material to inner layer, or lining, of hat 10.
24 Unobstructed, interlayer space between outer layer 14 and inner layer 16.
26 A slit in inner layer lining 16 providing an opening through which a hot or cold pack is inserted into the interlayer space 24.
28 An alternate, or extra, slit in outer layer 14 providing an opening through which a hot or cold pack is inserted into the interlayer space 24.
34 Lower edge of hat 10.
36 Upper edge of hat 10.
40 Hot or cold pack, small size.
42 Hot or cold pack, sized and shaped to fit about entire hear cavity.
44 Decorative dog ears.

It should be understood that the drawings are not necessarily to scale. In certain instances, details which are not necessary for an understanding of the present invention or which render other details difficult to perceive may have been omitted.

### DETAILED DESCRIPTION

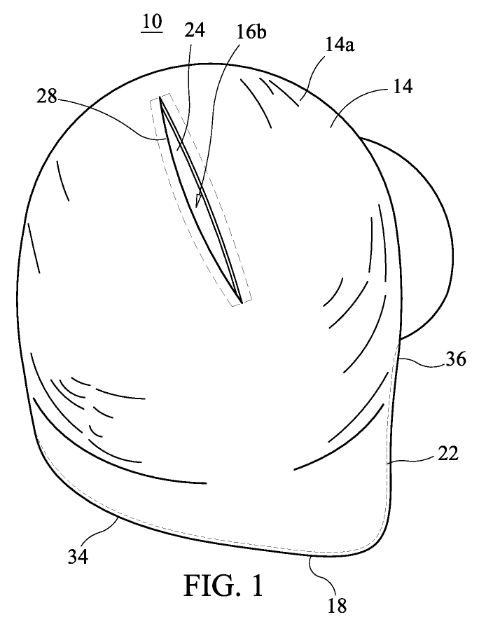

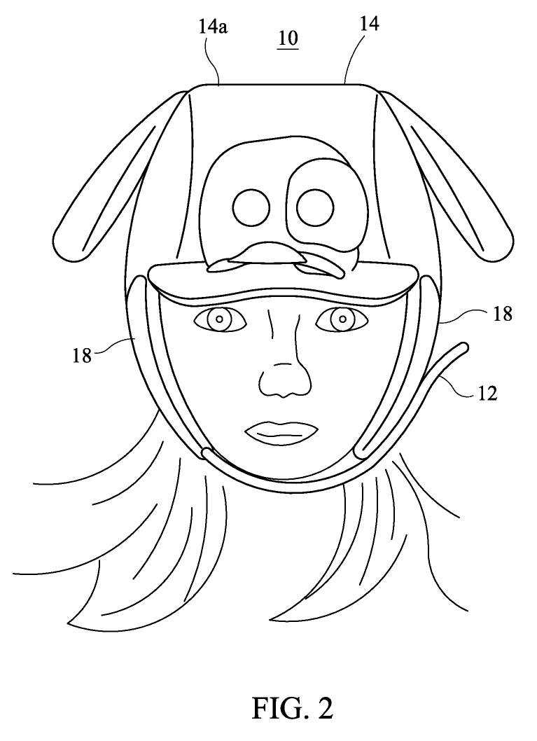

The present invention is directed towards a hat that provides for the application of hot or cold to any required part of a head. Turning now to the drawings, FIG. 1 illustrates a perspective view of a hat embodying the principles and concepts of the present invention and generally designated by the reference numeral 10.

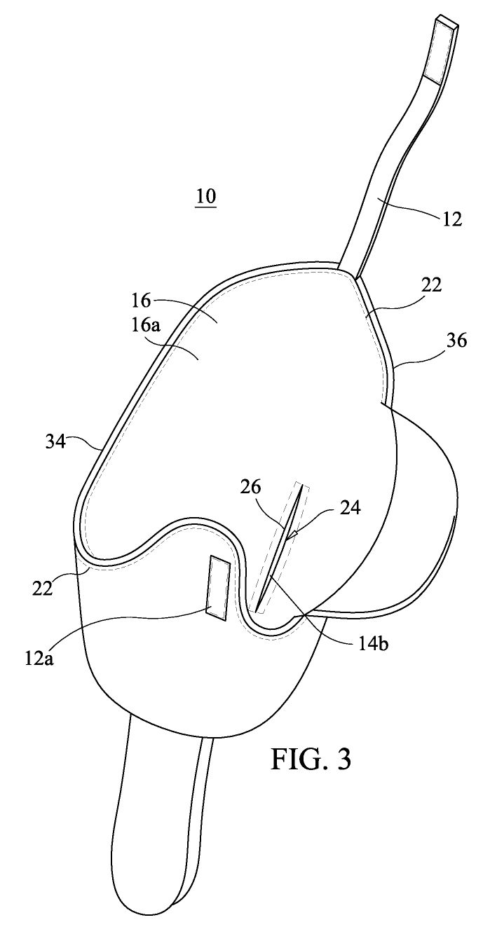

Hat 10 generally comprises an exterior layer 14 and an interior layer 16. Exterior layer 14 comprised outer surface 14a and inner surface 14b. Interior layer 16 comprised outer surface 16a and inner surface 16b, so that once exterior layer 14 and interior layer 16 are connected along the periphery of the hat, interlayer space 24 is created and is bounded by inner surface 14b and inner surface 16b. The interlayer space is an entirely open, unobstructed, accessible space between outer layer 14 and inner layer 16. In the example illustrated, exterior layer 14 is connected to interior layer 16 by sewing along the outer periphery of the finished hat forming seam 22. Hat 10 is sized and shaped to form a cavity adapted for snugly receiving the head of a wearer. It should be understood that sewing is just one way that exterior layer 14 may be connected to interior layer 16. The method used to connect the two layers does not define the invention. The layers could be, for example, connected together by heat gluing, adhesive, or by two-sided tape. The principles of the present invention require that exterior layer 14 is connected to interior layer 16 only about the periphery of the hat to ensure that the inner space thus formed has no seams preventing the targeted positioning of a hot or cold pack. Hat 10 comprises a lower edge 34 adapted for positioning to extend around the sides and back of a wearer's head including ear covering part 10 when the head of the wearer is received in the head cavity. Hat 10 also comprises upper edge 36 adapted to extend between opposite ends of lower edge 34 and around an upper periphery of the wearer's face including forehead covering part 20. Optionally, elongate strap 12 may be provided on hat 10 for securely affixing to the hat to a wearer's head, using, for example, connector 12a. Slit 26, in this example is positioned in interior layer 16 (see also FIG. 3), providing an opening through which a hot or cold pack is inserted into or removed from interlayer space 24. It is to be understood that slit 26 could alternatively, or also, be positioned in exterior layer 14 (as illustrated in FIG. 1). Having a silt in the exterior layer provides for positioning, adjusting, or replacing a hot or cold pack without having to remove the hat from the wearer's head, which is an important consideration if the hat is being worn by a sleeping infant or adult.

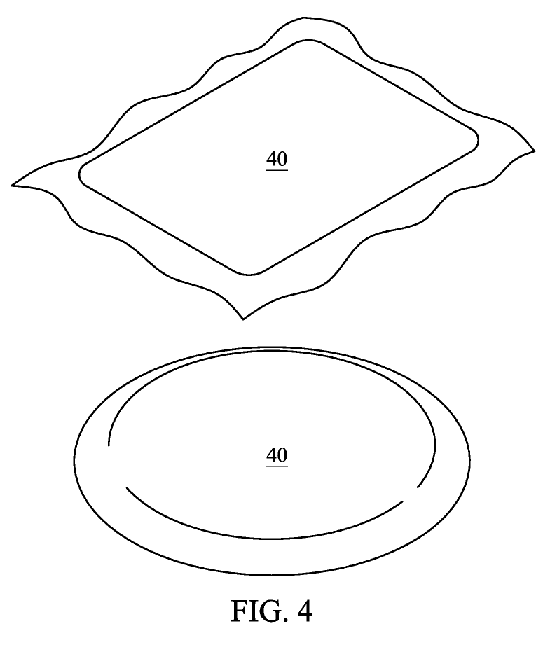

FIG. 4 illustrates two examples of hot or cold packs 40 provided according to the principles of the invention. Each pack 40 is easily and rapidly positioned in fully accessible interlayer space 24. Each of the packs contains a thermo-retentive material capable of being heated or cooled and of maintaining the heat or cool over a period of time. Heat retentive materials include gels, dry chemical compounds and organic materials. A pack is easily, rapidly, and removeably insertable into the interlayer space made accessible by either, or both, openings 26 and 28. Each of the packs is flexible to conform to the shape of a wearer's head. If desired a packet could be positioned within the interlayer space of the hat so that the entire hat, including pack, may be heated or cooled at the same time. In this case the materials from which the hat is made are suitable for use in a microwave for retaining heat generated in a microwave, or placement in a freezer.

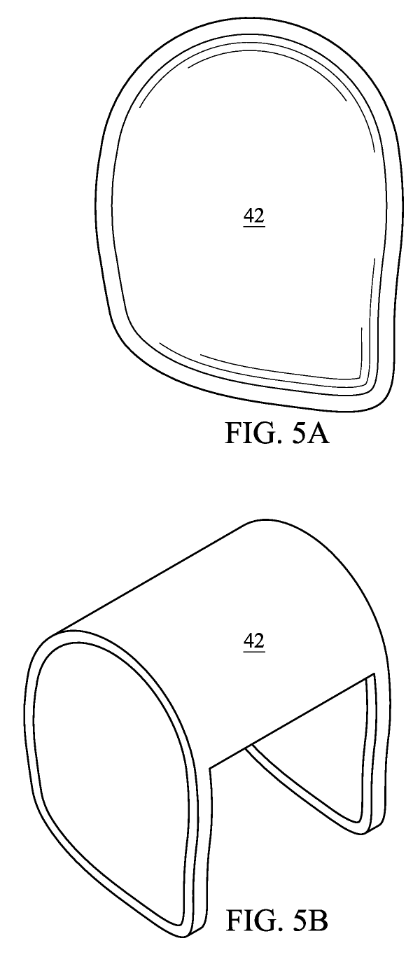

In the case the hat is to be used to apply heat or cold to a wearer's entire head, such as when heat is required for a none medical purpose, such as hair treatment, or for a medical requirement, such as the application of cold after head surgery has been performed, user has the option of placing several of the smaller packs 40 in various positions about the interlayer space of the hat; or of using hot or cold pack 42 that is made to fill the entire interlayer space, as is illustrated in FIGS. 5a and 5b.

A thermal treatment hat made according to the principles of the present invention is either of a simple, more mature design or is decoratively designed to be appealing to children. For instance, the hat may have ears 44 added to give the effect of a “dog-hat.”

In use, a user heats or cools the heat-retentive hot or cold pack and then, using a slit provided in the interior layer, places the pack(s) into the interlayer space of the hat so that the pack(s) will be directly over the spot to be heated or cooled, including the entire scalp, ear, and forehead area. The user would then place their head into the head cavity and secure the hat's strap under their chin, if the hat is provided with a strap. The heat from the containers would then radiate through the interior material layer to heat or cool the head area requiring treatment. In another embodiment, a user heats or cools the heat-retentive hot or cold pack and then, using a slit that is provided in the exterior layer, places the pack(s) into the interlayer space of the hat so that the pack will be directly over the spot to be heated or cooled. This embodiment is especially useful in those cases where it is difficult or inconvenient to remove a hat from the user's head to position, reposition, or to replace a pack. In yet an alternative embodiment, after positioning the pack(s) within the hat, the user would either heat or cool the thermo-retentive pack(s) while it is within the hat. In still yet another embodiment, a user would use a full-head covering thermal-retentive pack to place inside the hat. The full-head covering pack would be inserted in one or the other slits until it was correctly positioned. The full-head covering pack could be thermally treated either before placing it into the interlayer space of the hat or after. If desired, the hat is made with a slit through the interior material layer and/or through the exterior material layer.

With respect to the above description then, it is to be realized that the optimum dimensional relationships for the parts of the invention, to include variations in size, such as sized for adults or variously aged children, materials, such as insulating materials that will maintain heat or cold for longer periods and/or materials that lend themselves to a decorative theme, shape, for instance can be functional or fun, form, function, and manner of operation, assembly, and use, are deemed readily apparent and obvious to one skilled in the art, and all equivalent relationships to those illustrated in the drawings and described in the specification are intended to be encompassed by the present invention. The foregoing description, for purposes of explanation, uses specific and defined nomenclature to provide a thorough understanding of the invention. However, it will be apparent to one skilled in the art that the specific details are not required in order to practice the invention. Thus, it is to be understood that the foregoing description of the specific embodiment is presented for purposes of illustration and description and is not intended to be exhaustive or to limit the invention to the precise form disclosed. The invention is limited only by the claims.
What is claimed is:

1. A hat, comprising:
a hat made of two layers of material, comprising
an exterior layer sized and shaped to fit about the contours of a person's head,
an interior layer sized and shaped to fit about the contours of a person's head,
said exterior layer connected to said interior layer only along the periphery of the hat creating an unobstructed interlayer space for the insertion of a thermally retentive pack providing for the application of heat or cold to any specific area of a user's scalp, ears, and forehead.

2. The hat, as recited in claim 1, wherein said hat is shaped so as to cover said user's entire scalp, ears, and forehead area.

3. The hat, as recited in claim 1, wherein said interlayer space is shaped so as to cover said user's entire scalp, ears, and forehead area.

4. The hat, as recited in claim 1, wherein
said exterior layer further comprises
an exterior layer outer surface area and an exterior layer inner surface area, and
said interior layer further comprises
an interior layer outer surface area and an interior layer inner surface area,
so that said unobstructed interlayer space is bounded by said inner exterior layer surface area, said inner interior layer surface area, and a seam resulting from said exterior layer connected to said interior layer only along the periphery of the hat.

5. The hat, as recited in claim 1, wherein said exterior layer is connected to said interior layer by sewing.

6. The hat, as recited in claim 1, wherein said exterior layer is connected to said interior layer by adhesive bonding.

7. The hat, as recited in claim 1, wherein said exterior layer is connected to said interior layer so as to ensure that the interlayer space created by the connection has no seams creating barriers to the exact positioning of a hot or cold pack where it is required over a user's scalp, ear, or forehead area.

8. The hat, as recited in claim 1, wherein said hat further comprises a strap for securely affixing the hat to a user's head with at least one end of said strap attached to said hat.

9. The hat, as recited in claim 1, wherein said hat further comprises a slit throughout said interior layer so as to provide an opening through which a hot or cold pack is inserted into the interlayer space.

10. The hat, as recited in claim 1, wherein said hat further comprises a slit throughout said exterior layer so as to provide an opening through which a hot or cold pack is inserted into the interlayer space.

11. The hat, as recited in claim 1, wherein said hat further comprises a slit throughout said exterior layer and throughout said interior layer so as to provide an opening through which a hot or cold pack is inserted into the interlayer space.

12. The hat, as recited in claim 1, wherein said hat further comprises at least one thermal retentive pack for insertion into said interlayer space, said pack shaped and sized to be applied over a specific area of a head requiring treatment.

13. The hat, as recited in claim 1, wherein said hat further comprises at least one thermal retentive pack for insertion into said interlayer space, said pack shaped and sized to be applied over a user's whole head area.

14. The hat, as recited in claim 1, wherein said hat further comprises being made so as to provide for combined hat and thermal retentive pack cooling.

15. The hat, as recited in claim 1, wherein said hat further comprises being made having decorative animal features.

16. The hat, as recited in claim 1, wherein said inner surface of said exterior material layer and said inner surface of said interior material layer are made from a material having an amount of roughness required for said thermal retentive pack to be securely held in place solely by the frictional bond between the outer surfaces of the thermal retentive pack and the inner surfaces of said inner surface of said exterior material layer and said inner surface of said interior material layer.

17. The hat, as recited in claim 8, wherein said hat further comprises further structured so as to provide for a connector for connecting the end of said strap not attached to said hat to said hat.

18. A hat, comprising:
a hat made of two layers of material, comprising
an exterior layer sized and shaped to fit about the contours of a head,
an interior layer sized and shaped to fit about the contours of a head,
said exterior layer sewn to said interior layer only along the periphery of the hat creating an unobstructed interlayer space for the insertion of a thermal retentive pack providing for the application of heat or cold to all specific areas of a user's head, ear, and forehead areas,
said hat shaped so as to cover said user's entire scalp, ear, and forehead area,
said interlayer space shaped so as to cover said user's entire scalp, ear, and forehead area, having
a slit throughout said interior layer and/or said exterior layer so as to provide an opening through which a thermally retentive pack is inserted into the interlayer space and having,
a strap for securely affixing said hat to a user's head.

19. A hat, comprising:
a hat in combination with at least one thermally retentive pack, said hat made of two layers of material, comprising
an exterior layer sized and shaped to fit about the contours of a head,
an interior layer sized and shaped to fit about the contours of a head,
said exterior layer sewn to said interior layer only along the periphery of the hat creating an unobstructed interlayer space for the insertion of a thermal retentive pack,
said hat shaped so as to cover said user's entire scalp, ear, and forehead area,
said interlayer space shaped so as to cover said user's entire scalp, ear, and forehead area,
said hat having
a strap for securely affixing said hat to a user's head, and
a slit throughout said interior layer and/or said exterior layer so as to provide an opening through which said thermally retentive pack of a size and shape to be inserted into the interlayer space providing for the application of heat or cold to a discreet area of a user's head, ear, and forehead requiring heat or cold treatment.

20. The hat, as recited in claim 19, wherein said hat further comprises a thermally retentive pack sized and shaped to conform to a user's entire scalp area.
